(19) United States
(12) Patent Application Publication
(10) Pub. No.: US 2015/0086522 A1
(43) Pub. Date: Mar. 26, 2015

(54) Formulation containing herb infused aloe

(71) Applicant: MICHAEL TODD TRUE ORGANICS LP, PORT ST. LUCIE, FL (US)

(72) Inventor: Melanie Velez, Port St. Lucie, FL (US)

(73) Assignee: MICHAEL TODD TRUE ORGANICS LP, PORT ST. LUCIE, FL (US)

(21) Appl. No.: 13/987,996

(22) Filed: Sep. 23, 2013

(51) Int. Cl. A61K 31/122; A61K 36/886; A61K 45/06; A61K 31/01

(52) U.S. Cl. CPC A61K 31/122; A61K 31/01; A61K 36/886; A61K 45/06
USPC 424/94.1

(57) ABSTRACT

The present invention provides for a vitamin and herb infused aloe formulation which comprises aloe, at least one herb, at least one vitamin, at least one moisturizing agent, CoQ10 and Squalane.

### BACKGROUND OF INVENTION

1. Field of Invention

The present invention relates to a composition containing herb infused aloe. In particular, a formulation and related method of manufacture comprising: aloe, at least one herb, at least one vitamin, at least one moisturizing agent, CoQ10 and Squalane.

2. Description of Related Art

The prior art provides for numerous compositions containing aloe. The present invention provides for a delivery system that contains vitamin and herb infused aloe which retains its stability, functionality and aesthetics and optimizes performance.

### SUMMARY OF INVENTION

In one embodiment, the present invention provides for a composition comprising aloe, at least one herb, at least one vitamin, at least one moisturizing agent, CoQ10 and Squalane.

In another embodiment, the vitamin is selected from a group consisting essentially of Vitamin B, Vitamin B3, Vitamin B5, Vitamin C, Vitamin E or combinations thereof.

In yet another embodiment, the herb is selected from a group consisting essentially of Burdock, Burdock root, Lavender arnica, German chamomile, Calendula, Bilberry, Goldenrod, St. John's Wart, Echinacea, Eucalyptus, Neem, Carapa procera, Cedrela odorata, Sapele (Entandrophragma cylindricum) Utile, Sapele (Entandrophragma cylindricum) Sipo, Entandrophragma utile, Bossé (Guarea cedrata), Bossé (Guarea thompsonii), Khaya ivorensis (African Mahogany), Khaya senegalensis, Melia azedarach, Swietenia macrophylla (Big Leaf Mahagony), Toona australis, Toona ciliata, Toona sureni, Toona ciliata, Indian Lilac, Pennyroyal, Juniper berries, Wild ginger, Myrrh, Angelica, Comfrey, Horsetail, Gotu-Kala, Acai, Wheat germ, Sun flower, Safflower, Chamomile, Thyme, Rosemary, Lemon balm, Goldenseal, Feverfew, Licorice, Peppermint, Turmeric, Grape seed, Milk thistle, Ginseng, Evening primrose or combinations thereof.

In still yet another embodiment, the moisturizing agent is selected from a group consisting essentially of Glycosaminoglycan, Chondroitin, Heparin, Heparitin Sulfate, Hyaluronic Acid, Keratan Sulfate, Prohyal+®, Xilogel® or combinations thereof.

In a further embodiment, the aloe is aloe juice. In yet a further embodiment, the aloe is in liquid form. In still a further embodiment, the aloe is a dried powdered aloe. In yet a further embodiment, the dry powdered aloe is admixed with water to form aloe juice. In still yet a further embodiment, the herb is in powder form. In another further embodiment, the herb in the powder form is admixed with the aloe in liquid form.

In another embodiment, the aloe infused composition can be incorporated into cosmetics, pharmaceuticals, cosmeceuticals, nutriceuticals, creams, soaps, lotions, skin care products or in combination thereof.

In still another embodiment, the aloe is from about 10% to about 76% of the composition, the herb is from about 1.5% to about 8% of the composition, the vitamin is from about 1.5% to about 8% of the composition, the moisturizing agent is from about 1.5% to about 6% of the composition, the CoQ10 is from about 1.5% to about 6% of the composition, and the Squalane is from about 1.5% to about 6% of the composition.

In yet another embodiment, the present invention relates to a composition containing herbal infused aloe, and the composition comprises aloe and at least one herb, and the herb is selected from a group consisting essentially of Burdock root, Neem, Calendula, Lavender, Horsetail, Centella asiatica, Chamomile, Thyme, Goldenseal, Milk thistle or combinations thereof.

In still yet another embodiment, the composition comprises: aloe from about 10% to about 76%, Burdock root from about 1.5% to about 8%, Neem from about 1.5% to about 8%, Calendula from about 1.5% to about 8%, Lavender from about 1.5% to about 8%, Horsetail from about 1.5% to about 8%, Centella asiatica from about 1.5% to about 8%, Chamomile from about 1.5% to about 8%, Thyme from about 1.5% to about 8%, Goldenseal from about 1.5% to about 8%, and Milk thistle from about 1.5% to about 8% of the composition.

In a further embodiment, the composition further comprises at least one vitamin, and the vitamin is selected from a group consisting essentially of Vitamin B, Vitamin B3, Vitamin B5, Vitamin C, Vitamin E or combinations thereof.

In yet a further embodiment, the composition further comprises as least one anti-oxidant and the anti-oxidant is selected from a group consisting essentially of Acai, Alpha Lipoic Acid, Resveratol, Green Tea, Pomegranate, Vitamin C or combinations thereof. In still a further embodiment, the composition further comprises Squalane.

In still yet a further embodiment, the composition further comprises at least one moisturizing agent and the moisturizing agent is selected from a group comprising Glycosaminoglycan, Chondroitin, Heparin, Heparitin Sulfate, Hyaluronic Acid, Keratan Sulfate, Prohyal+®, Xilogel® or combinations thereof.

In another embodiment, the present invention relates to a method of manufacturing an herbal infused aloe product and the method comprises admixing at least one herb and at least one vitamin into aloe.

In yet another embodiment, the method further comprises admixing at least one Glycosaminoglycan into the product selected from a group consisting essentially of Chondroitin, Heparin, Heparitin Sulfate, Hyaluronic Acid, Keratan Sulfate and mixtures or combinations thereof.

In still another embodiment, the method comprises admixing CoQ10 into the product.

In still yet another embodiment, the method further comprises admixing Squalane into the product.

It is noted that there is no specific order for admixing the Glycosaminoglycan, CoQ10 and Squalane.

### DETAILED DESCRIPTION OF THE INVENTION

As required, detailed embodiments of the present invention are disclosed herein. It is to be understood that the disclosed embodiments are merely exemplary of the invention that may be embodied in various forms. Therefore, specific structural and functional details disclosed herein are not to be interpreted as limits but merely as a basis for teaching one skilled in the art to employ the present invention. The specific examples below will enable the invention to be better understood. However, they are given merely by way of guidance and do not imply any limitation.

In one embodiment, the present invention uses Organic Aloe Vera Juice which is obtained from the Aloe Vera Plant or “True or Genuine Aloe.” The Aloe Vera is a stemless or very short stemmed succulent plant with thick and fleshy, green leaves. The margin of the leaf is serrated and has small white teeth. In the summer, flowers are produced.

In cosmetic and skin care applications, Aloe has been noted to help increase the absorption of nutrients and vitamins by the body. Skin care ingredients are absorbed by the blood stream as is anything topically applied to the skin to the extent of its absorption.

In another embodiment, the present invention uses Burdock Root which are plants of the genus Arctium. The extracts of the Burdock Root are derived from the root of plant. Burdock has been used for centuries to treat a variety of ailments. Traditionally, it has been used as a “blood purifier” to clear the bloodstream of toxins, as a diuretic (helping rid the body of excess water by increasing urine output) and as a topical remedy for skin problems such as eczema, acne and psoriasis. Burdock root has also been noted as having anti-aging benefits per the Journal of Cosmetic Dermatology 2008 (Volume 7 Issue 4) due to it's anti-inflammatory properties.

In yet another embodiment, the present invention uses Neem (also known as Nimtree or Liliac Indian) which is a fast-growing evergreen tree in the mahogany family Meliaceae. The Neem used in the formulations of the present invention may be extract derived from leaves of the plant. Neem seeds, root, leaves and oil have been used for centuries for its anti-inflammatory and anti-microbial properties. It aids in the healing of wounds and skin repair by conditioning and protecting the skin. Neem oil contains immune-stimulating compounds that help the deeper skin layers fight pathogens below the skin surface. This helps skin remain supple. Face packs made from Neem bark soothe skin and the compounds are quickly absorbed into the skin, making skin feel soft and reducing the effects of aging.

In still another embodiment, the present invention uses Calendula. Calendula is a herbaceous plant in the daisy family Asteraceae. Calendula extracts used in the formulations of the present invention may be derived from flower of plant. Calendula has been shown to increase the rate of wound healing, heal rashes, act as an anti-inflammatory to the skin and increase skin hydration and firmness.

In still yet another embodiment, the present invention uses Horsetail (also known as “Snake grass’ or “Puzzle grass”) and is in the Equisetaceae family of vascular plants that reproduce by spores rather than seeds. There is strong scientific evidence of the antioxidant properties of horsetail extract.

In another embodiment, the present invention uses chamomile which is a daisy-like plant of the family Asteraceae. The chamomile extract used in the formulations of the present invention may be derived from flower of plant. Chamomile can also improve the appearance of skin, according to a study by L. Bauman published in the “Journal of Drugs in Dermatology” in November of 2007. The article states that chamomile can improve skin's texture and elasticity, as well as reduce signs of photo damage. It may have some antioxidant capacity and is used in skin care products as an emollient while providing anti-inflammatory benefits.

In a further embodiment, the present invention uses thyme which is an herb from the genus Thymus. The formulations of the present invention may use thyme derived from leaves of plant. Thyme is known to have anti-bacterial, anti-inflammatory and antioxidant properties. Thyme has been tested for its ability to kill acne-causing bacteria and is found to be very effective.

In yet a further embodiment, the present invention uses Goldenseal (also known as Orangeroot or Yellow puccoon) which is a perennial herb in the buttercup family Ranunculaceae. It has a thick, yellow knotted rootstock. The formulations of the present invention may use Goldenseal extract derived from flower of plant. Goldenseal is used in various skin formulations to increase wound healing, reduce eczema symptoms and improve the overall condition of skin affected by inflammatory conditions.

In still a further embodiment, the present invention uses Milk thistle which is a plant from the Asteraceae family. The formulations of the present invention may use milk thistle extract derived from flower of plant. Milk thistle's demulcent properties help to hydrate and soften dry skin, improving the texture, making it smooth and supple. Its inflammatory action calms redness and soothes irritation. Milk thistle also helps to clear symptoms associated with eczema.

In still yet a further embodiment, the present invention uses Lavender which is a flowering plant in the mint family, Lamiaceae. The formulations of the present invention may use milk thistle extract derived from flower of plant. Lavender has been found to improve acne, eczema and other skin conditions.

In another further embodiment, the present invention uses CoQ 10 (also known as Ubiquinone, Ubidecarenone or Coenzyme Q) which is a synthetic oil-like vitamin substance. CoQ 10 acts as an antioxidant to the skin, helps regenerate cells and inhibits UV induced photodamage of the skin.

In yet another further embodiment, the present invention uses Hyaluronic acid which is a natural substance derived from the comb of a rooster. Hyaluronic acid is an anionic, non-sulfated glycosaminoglycan found to increase the absorption of nutrients in the skin when applied topically. Hyaluronic acid also increases suppleness and hydration of the skin when topically applied. It is very effective at increasing skin hydration, thus improving the appearance of fine lines and wrinkles.

In still another further embodiment, the present invention uses Vitamin E which is a fat soluble compound. The formulations of the present invention may use Vitamin E derived from soybeans or sun flower seeds. Vitamin E is known to fight UV damage and acts as an antioxidant thus improving overall quality of the skin.

In still yet another further embodiment, the present invention uses Vitamin B3 Niacinamide which is a synthetic vitamin substance that improves the appearance of hyperpigmentation, inflammation of the skin and other signs of aging.

In another embodiment, the present invention uses Vitamin B5 which is made synthetically from d-pantolactone, and the formulation of the present invention may use Vitamin B5 derived from honey. When topically applied, it helps the skin retain moisture thus allowing for more supple and moisturized skin.

In still another further embodiment, the present invention uses Squalane a hydrocarbon and triterpene with the molecular formula C₃₀14₄₈. Due to the complete saturation of squalane, it is not subject to auto-oxidation making it desirable in cosmetic applications where it is used as an emollient and moisturizer The formulations of the present invention may use Squalene derived from plant extracts but also animal sources (shark liver oil). When topically applied, it aids the skin in retaining moisture, increases healing time and can reduce symptoms of various skin conditions such as psoriasis, eczema and an improve dry, dehydrated skin thus allowing for more hydrated, supple skin.

Tables 1-5 (below) set forth numerous embodiments of the present invention showing the ingredients and percentage of the ingredients relative to the formulation. The present invention is neither limited to these examples and tables nor these specific ingredients and percentages. Ingredients in other embodiments may be added or removed and percentages may also be changed, modified and altered.

TABLE 1

| Ingredients: | Percentage: |
|---|---|
| Aloe | 70% |
| Burdock | 2% |
| Neem | 2% |
| Calendula | 2% |
| Lavender | 2% |
| Hyaluronic acid | 2% |
| Vitamin E | 2% |
| Vitamin B3 | 2% |
| Vitamin B5 | 2% |
| Squalane | 2% |
| CoQ10 | 2% |
| Horsetail | 2% |
| Chamomile | 2% |
| Thyme | 2% |
| Goldenseal | 2% |
| Milk thistle | 2% |

TABLE 2

| Ingredients: | Percentage: |
|---|---|
| Aloe | 55% |
| Burdock | 3% |
| Neem | 3% |
| Calendula | 3% |
| Lavender | 3% |
| Hyaluronic acid | 3% |
| Vitamin E | 3% |
| Vitamin B3 | 3% |
| Vitamin B5 | 3% |
| Squalane | 3% |
| CoQ10 | 3% |
| Horsetail | 3% |
| Chamomile | 3% |
| Thyme | 3% |
| Goldenseal | 3% |
| Milk thistle | 3% |

TABLE 3

| Ingredients: | Percentage: |
|---|---|
| Aloe | 65% |
| Burdock | 2.3% |
| Neem | 2.3% |
| Calendula | 2.3% |
| Lavender | 2.3% |
| Hyaluronic acid | 2.3% |
| Vitamin E | 2.3% |
| Vitamin B3 | 2.3% |
| Vitamin B5 | 2.3% |
| Squalane | 2.3% |
| CoQ10 | 2.3% |
| Horsetail | 2.3% |
| Chamomile | 2.3% |
| Thyme | 2.3% |
| Goldenseal | 2.3% |
| Milk thistle | 2.3% |

TABLE 4

| Ingredients: | Percentage: |
|---|---|
| Aloe | 75% |
| Burdock | 1.6% |
| Neem | 1.6% |
| Calendula | 1.6% |
| Lavender | 1.6% |
| Hyaluronic acid | 1.6% |
| Vitamin E | 1.6% |
| Vitamin B3 | 1.6% |
| Vitamin B5 | 1.6% |
| Squalane | 1.6% |
| CoQ10 | 1.6% |
| Horsetail | 1.6% |
| Chamomile | 1.6% |
| Thyme | 1.6% |
| Goldenseal | 1.6% |
| Milk thistle | 1.6% |

TABLE 5

| Ingredients: | Percentage: |
|---|---|
| Aloe | 55% |
| Burdock | 3% |
| Neem | 2% |
| Calendula | 3% |
| Lavender | 3% |
| Hyaluronic Acid | 3% |
| Vitamin E | 4% |
| Vitamin B3 | 3% |
| Vitamin B5 | 3% |
| Squalane | 2% |
| CoQ10 | 3% |
| Horsetail | 3% |
| Chamomile | 3% |
| Thyme | 4% |
| Goldenseal | 3% |
| Milkthistle | 3% |

When used in conjunction with other skin formulations and ingredients, results are enhanced, as aloe formulation allows other skin ingredients to be absorbed by the skin more effectively. This formula acts as the base for other skin care ingredients and formulas. It also enhances the results of other skin care ingredients combined with this formula.

Testing was conducted regarding the performance of the formulations of the present invention. Product containing formulations of the present invention has been tested on group of 10 patients/subjects over a period of 90 days. Results were 85-90% improvement for each patient/subject in skin elasticity, appearance of wrinkles/fine lines, suppleness of skin and improvement in skin tone and texture over a period of 90 days. 85-90% reduction of inflammatory symptoms of the skin including rosacea and redness associated with acne or sensitive, redness prone skin in general have been noted through testing. For purposes of this invention, skin elasticity is defined as the ability of the skin to stretch, then return to its normal state or to firmness. For purposes of this invention, skin tone and texture are defined as color and feel of the skin.

The premise of the testing is that the formulations of the present invention with its specific blend of aloe juice along with various vitamins and plant extracts will be effective at allowing the absorption of the above ingredients/nutrients, allowing for a unique product to increase suppleness of the skin, allowing the skin to retain moisture, repairing skin cells and strengthening the structure of the skin in general.

Parameters for the Testing:

The formulation of the present invention was incorporated into a skin care formula and the formula was tested on a group of 10 individuals. The formula was applied to all areas of the skin of the face and neck twice daily every day for a period of 90 days. After 90 days, the face and neck regions were observed. It is noted that the 10 individuals had different skin conditions.

### Results:

Table 6 set forth below shows the results of the testing and the observations made after 90 days:

TABLE 6

| Test Subject | |
|---|---|
| Number: | Observations Before & After: |
| Patient/subject #1 | Patient's skin concerns and reason for application |
| | prior to testing were sensitivity and redness of the |
| | skin and lack of firmness and appearance of |
| | hydration of the skin. |
| | Improvement in skin suppleness, elasticity. |
| | Reduction in redness and sensitivity noted after |
| | testing. |
| Patient/subject #2 | Patient's skin concerns prior to testing were lack of |
| | firmness/elasticity of the skin and appearance of |
| | hydration of the skin. |
| | Improvement in skin suppleness, elasticity and |
| | firmness noted after testing. |
| Patient/subject #3 | Patient's skin concerns prior to testing were lack of |
| | appearance of hydration of the skin, appearance of |
| | fine lines and wrinkles, redness of the skin and |
| | sensitivity. |
| | Improvement in skin suppleness, reduction of |
| | appearance of fine lines and wrinkles, redness, and |
| | skin irritation/sensitivity noted after testing. |
| Patient/subject #4 | Patient's skin concerns prior to testing were |
| | discoloration of the skin, lack of firmness of the |
| | skin and appearance of fine lines and wrinkles. |
| | Improvement in skin tone and elasticity. Reduction |
| | of appearance in wrinkles and fine lines after |
| | testing. |
| Patient/subject #5 | Patient's skin concerns prior to testing were |
| | appearance of fine lines and wrinkles, appearance of |
| | hydration of the skin and lack of firmness of the |
| | skin. |
| | Reduction in appearance of fine lines, wrinkles, |
| | increased skin suppleness and firmness noted after |
| | testing. |
| Patient/subject #6 | Patient's skin concerns prior to testing were |
| | inflammation and redness of the skin due to acne, |
| | and lack of appearance of hydration of the skin. |
| | Reduction of acne, redness associated with acne, |
| | and improvement in skin suppleness of skin noted |
| | after testing. |
| Patient/subject #7 | Patient's skin concerns prior to testing were redness |
| | due to rosacea skin condition, inflammation and |
| | redness of the skin due to acne, and lack of |
| | appearance of hydration of the skin. |
| | Reduction in rosacea symptoms and redness. |
| | Reduction of acne and inflammation, and increased |
| | suppleness of skin noted after testing. |
| Patient/subject #8 | Patient's skin concerns prior to testing were |
| | appearance of fine lines and wrinkles, lack of |
| | firmness of the skin. |
| | Reduction in appearance of wrinkles, firmness of |
| | the skin noted after testing. |
| Patient/subject # 9 | Patient's skin concerns prior to testing were lack of |
| | firmness of the skin, appearance of fine lines and |
| | wrinkles and lack of appearance of hydration of the |
| | skin. |
| | Increased firmness of the skin, increased |
| | suppleness and reduction of wrinkles and fine lines |
| | noted. |
| Patient/subject #10 | Patient's skin concerns prior to testing were |
| | discoloration of the skin, inflammation due to acne |
| | as well as redness, and sensitivity of the skin due to |
| | inflammation. |
| | Improvement in skin tone and reduction of acne and |
| | redness/sensitivity associated with acne noted after |
| | testing. |

The conclusion through testing is that this formula, as a base for skin care products, enhances results and allows ingredients to be absorbed more effectively by the skin, thus improving the results when combined with other ingredients, allowing for improvement in various skin conditions.

The present invention is not limited to the above Examples and Tables. Numerous modifications and variations of the present invention are possible in light of the above teachings. It is therefore to be understood that within the scope of the attendant claims attached hereto, this invention may be practiced other than as specifically disclosed herein.

What is claimed is:

1. A composition comprising aloe, at least one herb, at least one vitamin, at least one moisturizing agent, CoQ10 and Squalane.

2. The composition of claim 1 wherein said vitamin is selected from a group consisting essentially of Vitamin B, Vitamin B3, Vitamin B5, Vitamin C, Vitamin E and combinations thereof.

3. The composition of claim 1 wherein said herb is selected from a group consisting essentially of Burdock, Burdock root, Lavender arnica, German chamomile, Calendula, Bilberry, Goldenrod, St. John's Wart, Echinacea, Eucalyptus, Neem, Carapa procera, Cedrela odorata, Sapele (Entandrophragma cylindricum) Utile, Sapele (Entandrophragma cylindricum) Sipo, Entandrophragma utile, Bossé (Guarea cedrata), Bossé (Guarea thompsonii), Khaya ivorensis (African Mahogany), Khaya senegalensis, Melia azedarach, Swietenia macrophylla (Big Leaf Mahagony), Toona australis, Toona ciliata, Toona sureni, Toona ciliata, Indian Lilac, Pennyroyal, Juniper Berries, Wild Ginger, Myrrh, Angelica, Comfrey, Horsetail, Gotu-Kala, Acai, Wheat germ, Sun flower, Safflower, Chamomile, Thyme, Rosemary, Lemon balm, Goldenseal, Feverfew, Licorice, Peppermint, Turmeric, Grape seed, Milk thistle, Ginseng, Evening primrose and combinations thereof.

4. The composition of claim 1 wherein said moisturizing agent is selected from a group consisting essentially of Glycosaminoglycan, Chondroitin, Heparin, Heparitin Sulfate, Hyaluronic Acid, Keratan Sulfate, Prohyal+®, Xilogel®, and combinations thereof.

5. The composition of claim 1 wherein said aloe is aloe juice.

6. The composition of claim 1 wherein said aloe is in liquid form.

7. The composition of claim 1 wherein said aloe is a dried powdered aloe.

8. The composition of claim 7 wherein said dry powdered aloe is admixed with water to form aloe juice.

9. The composition of claim 1 wherein said herb is in powder form.

10. The composition of claim 9 wherein said herb in said powder form is admixed with said aloe in liquid form.

11. The composition of claim 1 wherein said aloe infused composition can be incorporated into cosmetics, pharmaceuticals, cosmeceuticals, nutriceuticals, creams, soaps, lotions, skin care products and combinations thereof.

12. A composition of claim 1 wherein said aloe is from about 10% to about 76%, said herb is from about 1.5% to about 8%, said vitamin is from about 1.5% to about 8%, said moisturizing agent is from about 1.5% to about 6%, said CoQ10 is from about 1.5% to about 6%, and said Squalane is from about 1.5% to about 6% of said composition.

13. A composition containing herbal infused aloe, said composition comprising aloe and at least one herb, said herb is selected from a group consisting essentially of Burdock root, Neem, Calendula, Lavender, Horsetail, Centella asiatica, Chamomile, Thyme, Goldenseal, Milk thistle and combinations thereof.

14. The composition of claim 13 wherein
said Aloe from about 10% to about 76%,
said Burdock root from about 1.5% to about 8%,
said Neem from about 1.5% to about 8%,
said Calendula from about 1.5% to about 8%,
said Lavender from about 1.5% to about 8%,
said Horsetail from about 1.5% to about 8%,
said Centella asiatica from about 1.5% to about 8%,
said Chamomile from about 1.5% to about 8%,
said Thyme from about 1.5% to about 8%,
said Goldenseal from about 1.5% to about 8%, and
said Milk thistle from about 1.5% to about 8% of said composition.

15. The composition of claim 13 further comprising at least one vitamin, said vitamin is selected from a group consisting essentially of Vitamin B, Vitamin B3, Vitamin B5, Vitamin C, Vitamin E and combinations thereof.

16. The composition of claim 13 further comprising at least one antioxidant, said antioxidant is selected from a group consisting essentially of Acai, Alpha Lipoic Acid, Resveratrol, Green tea, Pomegranate, Vitamin C and combinations thereof.

17. The composition of claim 13 further comprising Squalane.

18. The composition of claim 13 further comprising at least one moisturizing agent, wherein said moisturizing agent is selected from a group comprising Glycosaminoglycan, Chondroitin, Heparin, Heparitin Sulfate, Hyaluronic Acid, Keratan Sulfate, Prohyal+®, Xilogel® and combinations thereof.

19. The composition of claim 13 wherein said aloe is aloe juice.

20. The composition of claim 13 wherein said aloe is in liquid form.

21. The composition of claim 13 wherein said aloe is a dry powdered aloe.

22. The composition of claim 21, wherein said dry powdered aloe is admixed with water to form aloe juice.

23. The composition of claim 13, wherein said herbs are in powder form.

24. The composition of claim 23, wherein said herbs in said powder form are admixed with said aloe in liquid form.

25. The composition of claim 13, wherein said composition of claim 13 can be incorporated into cosmetics, pharmaceuticals, cosmeceuticals, nutriceuticals, creams, soaps, lotions, skin products and in combination thereof.

26. A method of manufacturing an herbal infused aloe product, said method comprising admixing at least one herb and at least one vitamin into aloe.

27. The method of claim 26 wherein said aloe is aloe juice.

28. The method of claim 26 wherein said aloe is in liquid form.

29. The method of claim 26 wherein said aloe is a dry powdered aloe.

30. The method of claim 29 wherein said dry powdered aloe is admixed with water to form aloe juice.

31. The method of claim 26 wherein said herb is in powder form.

32. The method of claim 31 wherein said herb is in said powder form is admixed with said aloe in liquid form.

33. The method of claim 26 wherein said herb admixed into aloe product is selected from a group consisting essentially of Burdock, Burdock root, Lavender arnica, German chamomile, Calendula, Bilberry, Goldenrod, St. John's Wart, Echinacea, Eucalyptus, Neem, Carapa procera, Cedrela odorata, Sapele (Entandrophragma cylindricum) Utile, Sapele (Entandrophragma cylindricum) Sipo, Entandrophragma utile, Bossé (Guarea cedrata), Bossé (Guarea thompsonii), Khaya ivorensis, Khaya senegalensis, Melia azedarach, Swietenia macrophylla (Big Leaf Mahagony), Toona australis, Toona ciliata, Toona sureni, Toona ciliata, Indian lilac, Pennyroyal, Juniper berries, Wild ginger, Myrrh, Angelica, Comfrey, Horsetail, Gotu-Kala, Acai, Wheat germ, Sun flower, Safflower, Chamomile, Thyme, Rosemary, Lemon balm, Goldenseal, Feverfew, Licorice, Peppermint, Turmeric, Grape seed, Milk thistle, Ginseng, Evening primrose and mixtures thereof.

34. The method of claim 26 wherein said vitamin is selected from a group consisting essentially of Vitamin B3, Vitamin B5, Vitamin C and Vitamin E and mixtures thereof.

35. The method of claim 26 further comprising admixing at least one glycosaminoglycan into said product, said Glycosaminoglycan selected from a group consisting essentially of Chondroitin, Heparin, Heparitin Sulfate, Hyaluronic Acid, Keratan Sulfate and mixtures thereof.

36. The method of claim 26 further comprising admixing CoQ10 into said product.

37. The method of claim 26 further comprising admixing Squalane into said product.
